(19) United States
(12) Patent Application Publication
(10) Pub. No.: US 2004/0147458 A1
(43) Pub. Date: Jul. 29, 2004

(54) Compositions to reduce exercise-induced swelling of joints

(76) Inventors: Richard K Van Tassel, Porterville, CA (US); Terri L. Butler, Kirkland, WA (US); John St. Cyr, Coon Rapids, MN (US)

Correspondence Address: Schwegman Lundberg, Woessner & Kluth, PO Box 2938, Minneapolis, MN, 55402, US

(21) Appl. No.: 10/473,693

(22) PCT Filed: Mar. 28, 2002
(86) PCT No.: PCT/US02/09941

(51) Int. Cl. A61K 31/70

(52) U.S. Cl. 514/23

(57) ABSTRACT

Ribose, given before and preferably also after strenuous exercise prevents or treats joint swelling due to strenuous exercise.

### FIELD OF THE INVENTION

This invention is directed to the prophylaxis and therapy of exercise-induced swelling of joints in mammals such as humans, horses and dogs.

### BACKGROUND OF THE INVENTION

Mammals, such as humans, horses and dogs, are subjected to acute trauma during exercise whether it be for training, competitive events or for therapy. The average speed of racing Thoroughbreds is about 1050 meters per minute. Racing greyhounds run at about fifteen miles per hour and human marathoners can run 26 miles in just over two hours. Such speed is accompanied by a high metabolic rate, a high respiration rate and stresses to articular areas. Even in less fit mammals, increased exercise that is far short of the speed of the athlete can cause wear and tear to the joints, resulting in swelling.

The continuous pounding of the leg joints results in acute swelling of the joint area, which is thought to be an acute trauma inflammatory response due to tissue injury. The trauma may cause membrane disruption and tears in the blood vessels, ligaments and tendons surrounding the joint. The extruded cell contents induce white blood cell infiltration. This sequence of events results in edema, which is often accompanied by pain as the swollen tissues impinge on nerves or as a direct response to the cytokines released by the white blood cells.

The swelling can be relieved by various known methods. Generally, cold packs are applied when swelling is noted, a process which is somewhat difficult, labor-intensive and requiring specially designed packs to fit the mammal's joint. Elevation of the swollen joint may also offer a benefit. It is well-known to inject anti-inflammatory steroids such as prednisone directly into inflamed joints. However, steroid injection, while providing near term relief can actually lead to arthropathy. A hyaluronic acid preparation for intra-articular administration for treating inflammation of joints and steroid-induced arthropathy has also been recommended. U.S. Pat. No. 5,916,565 discloses the use of chondroitin sulfate and glucosamine to treat joint disorders in vertebrates. The composition is directed toward chronic joint disorders accompanied by destruction of cartilage, rather than to acute trauma reactions, which is the case with otherwise healthy exercising mammals. It is not known if this composition would be effective as a prophylaxis. An oral composition of N,N-dimethyl-glycine is disclosed in U.S. Pat. No. 5,026,728 as beneficial for treating inflammation. Subjection to a magnetic flux and application of ultrasound have also been suggested to be of benefit in joint swelling. Oral steroids such as prednisone, ingestion of aspirin and NSAID medication have also been recommended. Whatever the therapy, the mammal is generally allowed a rest period until the swelling diminishes.

The known methods are therapeutic, rather than prophylactic, and many are labor-intensive. The need remains for a composition that will decrease the results of this acute trauma on the joints of exercising mammals before swelling occurs and that will decrease the length of time necessary to recover from any residual swelling.

### SUMMARY OF THE INVENTION

Ribose is given to a mammal before and/or after strenuous exercise, preferably in a form that is quickly absorbed into the blood stream, in order to reduce swelling in joints and to minimize recovery time from any swelling that does occur. An effective dose of ribose is 65 to 300 mg/kg body weight. When the mammal is a horse, the preferred dose is 30 to 200 gm. A more preferred dose is 40 to 100 gm ribose. When the mammal is a dog, the preferred dose is two to ten gm ribose. When the mammal is a human, the preferred dose is one to 20 gm ribose. The ribose is administered within one half hour of the strenuous exercise and the mammal should not have eaten sufficient food to interfere with the rapid absorption of the ribose.

### DETAILED DESCRIPTION OF THE INVENTION

As is disclosed in U.S. Pat. No. 6,159,942, ribose is beneficial to raise basal ATP levels in muscle, thus enhancing energy levels. U.S. Pat. No. 6,159,943 discloses that ribose is beneficial to prevent or relieve muscle aches and cramping. In pending patent application Ser. No. 09/677634, filed Oct. 3, 2000, it is disclosed that ribose administered to athletes, including race horses and racing dogs, as a dietary supplement enhances athletic performance by reducing fatigue. All of these references disclose the beneficial effects of ribose on skeletal muscle. This invention discloses that the administration of ribose surprisingly prevents or alleviates swelling around joints due to trauma to the ligaments, tendons and blood vessels surrounding the joint. Unlike skeletal muscle, these tissues have comparatively low rates of ATP utilization and reduced circulation. It is therefore not expected that the administration of ribose would have an effect on preventing or alleviating swelling.

The suggested dosage for a human is three to 20 grams daily; for a horse, 30 to 300 gm daily and for a dog, 0.5 to 10 gm daily. This dose is most easily given as a supplement in tablets or capsules, drinks, bars, mixed into feed, added as topfeed or given intravenously. As will be seen from the following examples, those mammals taking ribose as a supplement to enhance physical performance will not obtain the benefits of the present invention unless the disclosed protocols are followed.

Mammals experiencing joint swelling and pain due to strenuous exercise will benefit from the methods of this invention. By strenuous exercise is meant running, wherein the knee joints suffer pounding and concomitant trauma. For humans, strenuous exercise can also include heavy lifting, wherein both the knee and shoulder joints may swell and also racket games, where the elbow joints may be affected. By ribose is meant D-Ribose.

The following examples are provided as preferred embodiments of the invention and are not to be considered as limiting the scope of the attached claims. Insofar as the references cited may help to understand the invention, the teachings thereof are hereby incorporated by reference.

### EXAMPLE 1

### Racing Stable of 12 Horses

Applicant's suggested dietary supplement for horses contains, in addition to ribose, glucosamine, chondroitin sulfate, dimethylglycol, vitamins and minerals. It was discovered that horses fed this supplement showed less swelling after races than the same horses had shown before ribose supplementation. Experimentation with ribose alone in different dosages and timing of doses showed surprisingly that additional benefit was seen when an easily absorbed dose of ribose alone was given immediately before and immediately after each race.

There are several known ways in which to deliver an easily absorbed dose to a horse. Drenching is a common means of delivering doses directly to the horse's stomach. A gastric tube is inserted through the horse's nostril and a solution is passed through the tubing by gravity. Alternatively, ribose can be given in a small amount of drinking water. Since it is mildly sweet, the taste is attractive to a horse. A paste can be applied directly into the horse's mouth, which may allow rapid buccal absorption. The ribose may also be administered intravenously. The mode of application is not important to the practice of the invention providing that (1) the osmolarity of the dose is hypotonic, (2) ribose is the main ingredient, and (3) the ribose is not given with other food, which would delay absorption.

The dosage given should be adjusted carefully to each animal. It has been observed that the swelling reaction is variable; some horses have a great deal of swelling after each race and others are less affected. Ribose at suggested levels causes no side-effects, but for cost considerations, it is desirable to determine for each horse the lowest effective dose. Beginning with a dose of 20 gm ribose, the dosages were adjusted upward. It was found that a dose 50 to 60 gms of ribose was optimal for most horses. Although the weight of the horse was not a factor, the state of nourishment was important. Less well-nourished horses require less ribose for the prophylaxis and therapy of joint swelling. In those horses tested by Applicant, which had been maintained on the ribose-containing supplement, it was seen that a dosage of about 100 or 200 grams of ribose was required to prevent or reduce swelling. In all mammals it may be seen that increasing doses of ribose are necessary to provide the benefit of this invention. This seeming paradox may be due to the fact that mammals receiving ribose as a maintenance dietary supplement have more energy, run faster and thus sustain more acute exercise-related trauma to the joints. It must be stressed that whether or not horses are maintained on a ribose-containing dietary supplement such as that of Applicant, in order to obtain the benefits of this invention, the dosage and timing disclosed above must be followed.

It is also important to continue administering ribose before and after each race in order to decrease the interval between races or the period of rest necessary after each race. It is also desirable to administer ribose if a particularly hard workout is planned. It is expected that the use of ribose to prevent swelling may prevent the acute joint trauma from progressing to chronic swelling or arthritis, thus lengthening the racing life of a horse.

Twelve Standard Bred horses, three to eight years of age, were tested. All of the animals had been observed to show joint swelling after racing. The swelling was treated either with cold packs or rest. Time to recovery was approximately one hour to several days. In general, the horses were rested for three days and then exercised by jogging each day, running two miles and then increasing the miles to six to eight.

The test horses were put on a schedule of ribose administration in a dosage of 40 gm before each race. Although some benefit was observed, most of the animals still showed some joint swelling. Therefore, the standard procedure was to administer the optimal dose of ribose both before and after each race. Following ribose administration, the majority of the animals showed no swelling and no further therapy was performed. About one-third of the animals showed moderate swelling and cold packs were applied. Time to recovery of these animals was about ten hours. These animals were able to resume training earlier than those not given ribose before and after racing. A subjective measure of benefit was seen in the attitude of the horses. Even with the ribose given only before the race, the horses were observed to be willing to play and move around their stalls, while usually a horse will remain quietly in the back of the stall after a race.

### EXAMPLE 2

### Horses Requiring Higher Levels of Ribose

Those horses who did not show complete prevention of swelling with ribose administration were administered higher levels of ribose. After the horses had been on ribose maintenance for a time, all required higher doses of ribose, up to 100 gm a dose given both before and after racing, in order to show a benefit.

### EXAMPLE 3

### Racing Dogs

Racing dogs, generally greyhounds, are also susceptible to swollen joints after racing. Administration of the proper dosage of ribose is expected to give the same benefit as is shown for horses. The observed dosage for a horse was approximately 100 to 300 mg per kilogram body weight. Based on this observation, a greyhound weighing 65 pounds will benefit from a dose of about two to twelve grams of ribose which is approximately 65 to 300 mg per kilogram. The dosage should be given just before each race or hard workout. While some incremental benefit may be seen if ribose is given after a race also, it has been found that a high dose before the race is adequate in most cases to prevent or reduce joint swelling. Administration can be by any convenient means. Ribose has a slightly sweet taste and dogs will readily drink a solution of ribose in water. Alternatively, the ribose solution can be sprayed into the dog's mouth for buccal absorption.

Applicant has found that a preferred method of administration is the placement of a bolus of a gel preparation containing ten grams of ribose delivered into the dog's mouth by syringe just before the race is particularly convenient. As for horses, the ribose should not be given more than half an hour before the race or with other food or anything else that would delay absorption.

### EXAMPLE 4

### Humans

Humans who run fast or for long distances, whether in competition or for recreation, sometimes suffer sufficient pounding of the knees and ankle to cause swelling. A human experiencing that condition can self-administer ribose in any convenient form, preferably dissolved in a small amount of water to be drunk, before and preferably after each run. The recommended unit dosage is one to 20 gm ribose.

Non-runners can also experience swelling in the joints. One subject who works in construction and does a lot of lifting frequently has swollen knees with accompanying soreness. The soreness subsides after a dose of about five grams of ribose. A heavy equipment operator has suffered from soreness in his knees for several years following an injury to the joint. A five gram dose of ribose is effective to lessen the achiness. The third subject is a competitive “strong man” who competes in such trials as lifting heavy rocks and pulling buses. In the course of his training and competition, he has stressed his knee joints, which are frequently swollen and sore. He takes doses of five to ten grams of ribose and finds that his symptoms are lessened. On one occasion, he ingested a dose of 30 grams of ribose and the soreness in his knees disappeared within twenty minutes.

Humans who exercise other joints, particularly the elbow and shoulder, to the point of pain and swelling also can prevent and treat swollen joints by administering ribose before and, preferably, also after the exercise. This group of humans includes tennis and golf players and manual workers.

Humans who are less fit will incur exercise-induced swelling of the joints at levels of exercise that would be considered extremely mild by humans who are younger or more fit. It is common for cardiac patients or obese patients who have been sedentary to be put on an exercise program. Treadmills, stationary bicycles and stair climbing machines are commonly used for exercise for this group, as well as walking. Although the levels of activity are low, joint swelling is common. Additionally, persons who have been bedridden for a time, as for recovery from trauma or major surgery, may find that they experience swollen joints when they resume exercising. All of these groups will benefit from the administration of ribose around the time of exercise.

We claim:

1. A method for preventing exercise-induced swelling of joints of a mammal comprising the administration of an effective amount of ribose immediately before the exercise that causes joint swelling.

2. A method for treating exercise-induced swelling of joints of a mammal comprising the administration of an effective amount of ribose immediately after the exercise that causes joint swelling.

3. A method for preventing and treating exercise-induced swelling of joints of a mammal comprising administration of an effective amount of ribose immediately before and immediately after the exercise that causes joint swelling.

4. The methods of claims 1, 2 or 3 wherein the effective amount of ribose is from 65 to 300 mg/kg body weight.

5. The methods of claims 1, 2 or 3 wherein the effective amount of ribose is from 100 to 200 mg/kg body weight.

6. A method of preventing exercise-induced swelling of the leg joints of a horse comprising the administration of from 30 to 100 gm of ribose immediately before a race.

7. A method of treating the exercise-induced swelling of the leg joints of a horse comprising the administration of from 30 to 100 gm of ribose immediately after a race.

8. A method of preventing the exercise-induced swelling of the leg joints of a dog comprising the administration of from one to ten gm of ribose immediately before a race.

9. A method of treating the exercise-induced swelling of the leg joints of a dog comprising the administration of from one to ten gm of ribose immediately after a race.

10. The methods of claims 6, 7, 8 or 9 wherein the ribose is administered from ten to 30 minutes before or after a race.

11. A method of preventing the exercise-induced swelling of the leg, shoulder or elbow joints of a human comprising the administration of from one to 30 gm of ribose within ten to 30 minutes prior to the exercise.

12. A composition for treating or preventing exercise-induced swelling of the joints of a mammal comprising ribose.

13. Use of ribose for the manufacture of a medicament for treating or preventing exercise-induced swelling of the joints of a mammal.
